(19) United States
(12) Patent Application Publication
(10) Pub. No.: US 2009/0148958 A1
(43) Pub. Date: Jun. 11, 2009

(54) NUCLEOTIDE-TRANSITION METAL COMPLEX CATALYST

(75) Inventor: Akon Higuchi, Kanagawa (JP)

Correspondence Address: STITES & HARBISON PLLC, 401 COMMERCE STREET, SUITE 800, NASHVILLE, TN, 37219, US

(73) Assignee: OneCell Inc.

(21) Appl. No.: 12/298,732

(22) PCT Filed: Apr. 27, 2007
(86) PCT No.: PCT/JP2007/059244

§ 371 Date: Oct. 27, 2008

(30) Foreign Application Priority Data
Apr. 27, 2006 (JP) 2006-122956

(51) Int. Cl. G01N 33/566; C07H 21/02; C07F 15/00

(52) U.S. Cl. 436/501; 536/23.1; 556/136

(57) ABSTRACT

This invention is to provide a catalyst (an artificial enzyme) which can be used as an alternative to a protein enzyme in the field relating to medicine, pharmaceuticals, biochemistry or chemical engineering. Such a catalyst comprises a complex of a transition metal and a monomeric or polymeric nucleotide or an analogue thereof.

### TECHNICAL FIELD

The present invention relates to a catalyst which comprises a complex of a transition metal and a nucleotide or an analogue thereof.

### BACKGROUND ART

Various chemical reactions, including metabolism, in living bodies are facilitated by biocatalysts, i.e., enzymes. Enzymes are made of proteins, which are polymers of amino acids. Typical examples of enzymes are glucose oxidase, peroxidase, urease, alcohol dehydrogenase, protease, amylase, glycogen phosphatase and the like. It was common knowledge that such biocatalysts are made of proteins.

However, nucleic acids, i.e., ribonucleic acids (RNA) and deoxyribonucleic acids (DNA), have been found to have enzymatic activity, i.e., catalytic activity, in recent years. For example, ribosomes synthesizing proteins are composed of RNA and protein, and these ribosomal RNAs play a main role in protein synthesis. It is generally believed that biocatalysts facilitating chemical reactions were RNAs in the early stages of the evolution of living organisms, and RNAs have been replaced with proteins in the process of the evolution.

Thus, RNA and proteins have been used as biocatalysts in living bodies, while DNA has not developed as a biocatalyst in the process of the evolution. It is believed that this is because such complementary pair structures as those of DNA cannot form complicated, catalytic active sites. It is also believed that the chemical stability of DNA prevents its structural evolution leading to acquisition of catalytic activity.

However, DNA enzymes (deoxyribozymes) performing the cleavage of intracellular messenger RNA and DNA modification reactions have been developed though recent advances in DNA engineering (Patent reference 1, Patent reference 2, Patent reference 3, Non-patent reference 1, and Non-patent reference 2). These DNA enzymes (deoxyribozymes) are expected to be used as medicines suppressing genes based on the degradation of intracellular messenger RNA and as parts of diagnostic kits.

As a DNA with catalytic activity, for example, a small DNA enzyme known as “10-23” performs the site-specific cleavage (hydrolysis reaction) of RNA with an extremely high reaction rate of 10 min⁻¹ (Non-patent reference 2). DNA enzymes based on this “10-23” model are called as “10-23 DNAzyme”, and has a catalytic domain of 15 deoxyribonucleotides, flanked by two substrate-recognition domains. By in vitro analysis, this type of DNA enzyme (DNAzyme) is shown to cleave substrate RNAs at purine-pyrimidine junctions efficiently at physiological conditions (Non-patent reference 3).

Y. Li and R. R. Breaker (Non-patent reference 4), J. Haseloff and W. L. Gerlach (Non-patent reference 5), and Raillard, S. A. and Joyce, G. F. (Non-patent reference 6) describes technologies that amplify the catalytic activity exhibited by the above DNA alone by using metal ions or cofactors of small molecules, or modification with functional groups, respectively.

JP-2005-517409-A (Patent reference 1) discloses a technology of de novo fluorescence-generating RNA-cleaving DNA enzyme system, which is capable of generating a very large fluorescent signal upon RNA cleavage, and exhibits a very large catalytic rate constant. This deoxyribozyme catalyzes site-specific RNA cleavage (hydrolysis reaction).

JP-2003-267990-A (Patent reference 2) discloses deoxyribozymes made of novel oligonucleotides, which contain as a constitutional unit a deoxyribonucleotide residue having a peptide group as a base moiety. This deoxyribozyme catalyzes site-specific cleavage of RNA.

JP-2003-506078-A (Patent reference 3) discloses deoxyribozymes, which are targeted against mRNA molecules encoding a subunit of the transcription factor NF-κB and specifically cleave Re1A (p65) mRNA.

It has been reported that a DNA aptamer recognizing three dimensional structures was prepared to recognize hemin with a protoporphyrin backbone, and the peroxidase-like catalytic activity of the hemin was investigated (Non-patent reference 7, 8). However, hemin has a structure where iron is surrounded by and bound to the protoporphyrin as a ligand, and it does not form a complex with DNA. Furthermore, since hemin molecule itself reacts with luminol which is a substrate for peroxidase activity, the activity of the DNA aptamer should be attributed to a catalytic reaction by hemin rather than DNA.

It has not yet been reported that DNA bound to a transition metal complex exhibits catalytic activity which is not exhibited by the DNA alone, the transition metal complex alone, or a ligand of the complex alone.

On the other hand, as to RNA, group 1 ribozyme splicing RNA is known to facilitate the reaction (1013 times), as compared to under physiological conditions. Many RNA enzymes (Ribozymes) are reported to exhibit higher activity than protein enzymes (Non-patent reference 5), and shown to be able to cleave both RNA and DNA molecules.

It has been reported that the hydrolysis of RNA by ribonuclease A (RNase A) is initiated by histidine at position 12, which acts as a base catalyst to deprotonate the 2′-OH group and thereby forming a 5 ligands-bound, transition state intermediate with phosphorus atom (Non-patent reference 9). It is also evident that histidine at position 119 acts as an acidic catalyst to cleave the exocyclic P-(5′-O) bond, located out of ring, and to protonate the released 5′-oxygen.

Hammerhead type RNA enzyme (ribozyme) catalyzes the specific cleavage of RNA molecules, and is able to cleavage complementary RNA substrates into its cis- and trans-forms (Patent reference 4). JP-2003-289866-A (Patent reference 5) discloses a technology of a novel gene relating to TNF-a induced apoptosis and a ribozyme inhibiting its expression.

However, it has not yet been reported that RNA bound to a transition metal complex exhibits catalytic activity which is not exhibited by the RNA alone, the transition metal complex alone, or a ligand of the complex alone.

Also, it has not yet been reported that nucleotide monomer or oligomer (e.g., in the order of 5 to 20-mer) bound to a transition metal complex exhibits enzymatic activity which is not exhibited by the nucleotide alone, the transition metal complex alone, or a ligand of the complex alone.

Since some platinum-complexes, represented by cisplatin, bind to DNA, and exert anticancer effect, many researches have been performed on DNA-platinum complexes. It has been generally reported that the reaction of the transition metal complex cisplatin and its analogs with DNA under physiological conditions is mainly based on binding of N-7 of guanine base of the DNA to the platinum, due to leaving chloride atom in the cisplatin and analogs (Non-patent reference 10 and Non-patent reference 11). Cisplatin can also react with N-1 of adenine base and N-3 of cytosine base.

Many methods for labeling a nucleic acid with a transition metal complex, especially a nucleic acid-platinum complex, have also been reported (e.g., Patent references 6-13). The labeled nucleic acids have been used as labeled probes to bind DNA and proteins. Nucleic acid-platinum complex having a fluorescent dye or vitamin as a ligand has been used for the detection of DNA and trace amount components in living bodies (e.g., Patent reference 6 and Patent reference 7). In the nucleic acid-transition metal complexes and nucleic acid-platinum complexes reported in these references, a component independently having catalytic activity such as protein enzyme, or fluorescent component, chemiluminescent component, or detectable moiety such as antibody was added. None of the references describes or suggests that the nucleic acid-transition metal complex exhibits catalytic activity that is not exhibited by the nucleic acids alone, the transition metal complex alone, or a ligand of the complex alone. It is not described or suggested that the nucleic acid-platinum complex exhibits catalytic activity that is not exhibited by the nucleic acids alone, the platinum complex alone, or a ligand of the complex alone.

Platinum metal is widely used as a reduction catalyst for chemical reactions in chemical synthesis, as an oxidation catalyst for removal of incomplete combustion gases from automobile exhaust gases, and the like. In these catalytic reactions, the catalytic action is thought to be exerted as follows: on the surface of the platinum metal, the respective atoms of hydrogen molecule, nitric monoxide molecule or the like are paired with platinum metal atoms to individually absorb with them, thereby the molecule takes an activated state where the intermolecular bonding force is weaken. The adjacent alignment of the metal atoms is essential for the catalytic function. Thus, the metal complex with the central metal being surrounded by the ligands is different in structure from the metal.

Conventionally, protein enzymes have been used as a means for detection and quantification of trace amount of components, such as nucleic acids (DNA, RNA), proteins, peptides, vitamins, amines, in living bodies and chemically synthesized substances (e.g., pharmaceuticals and agricultural chemicals). Enzymes such as peroxidase, alkaline phosphatase and the like have been used after conjugating them to binding partners, such as antibody, avidin, protein A/G, lectin, complementary nucleic acids and the like, that can specifically bind to target substances.

However, there were problems that conventional protein enzymes lose their catalytic activity during long storage because of their low chemical stability, or that they were not suitable for using at high temperature.
Patent reference 1: JP-2005-517409-A
Patent reference 2: JP-2003-267990-A
Patent reference 3: JP-2005-506078-A
Patent reference 4: JP-2000-511428-A
Patent reference 5: JP-2003-289866-A
Patent reference 6: JP-2001-521511-A
Patent reference 7: JP-3088287-B
Patent reference 8: JP-2004-129659-A
Patent reference 9: JP-2001-503742-A
Patent reference 10: JP-2004-129659-A
Patent reference 11: JP-2003-102499-A
Patent reference 12: JP-2005-522405-A
Patent reference 13: EP-1373572-B
Non-patent reference 1: R. R. Breaker, Molecular Biology: Making Catalytic DNAs, Science, 290(5499), 2095-2096 (2000).
Non-patent reference 2: D. Sen, C. R. Geyer, DNA enzymes, Curr. Opin. Chem. Biol., 2(6), 680-687 (1998).
Non-patent reference 3: S. W. Santoro, G. F. Joyce, A general purpose RNA-cleaving DNA enzyme, Proc. Natl. Acad. Sci. USA, 94, 4262-4266 (1997).
Non-patent reference 4: Y. Li, R. R. Breaker, Deoxyribozymes: New players in the ancient game of biocatalysis, Curr. Opin. Struct. Biol., 9(3), 315-323 (1999).
Non-patent reference 5: A. Jaschke, Artificial ribozymes and deoxyribozymes, Curr. Opin. Struct. Biol., 11(3), 321-326 (2001).
Non-patent reference 6: G. M. Emilsson, R. R. Breaker,
Deoxyribozymes: new activities and new applications, Cell Mol. Life. Sci., 59(4), 596-607 (2002).
Non-patent reference 7: Y. Tian, Y. He, C. Mao, Cascade signal amplification for DNA detection, ChemBioChem, 7, 1862-1864 (2006).
Non-patent reference 8: Y. Ito, Hasuda H., Immobilization of DNAzyme as a thermostable Biocatalyst, Biotechnology and Bioengineering, 86, 72-77 (2004).
Non-patent reference 9: Haseloff, J. Gerlach, W. L. Simple RNA enzymes with new and highly specific endoribonuclease activities, Nature, 334, 585-591 (1988).
Non-patent reference 10: Suzanne E, Sherman and Stephen J. Lippard, Structural aspects of platinum anticancer drug interactions with DNA, Chem. Rev., 87(5), 1153-1181 (1987).
Non-patent reference 11: Temple, M. D. et al., Interaction of Cisplatin and DNA-Targeted 9-Aminoacridine Platinum Complexes with DNA, Biochemistry, 39(3), 5593-5599 (2000).

### DISCLOSURE OF THE INVENTION

### Problems to be Solved by the Invention

There has been a desire for a catalyst (artificial enzyme) which can be used as an alternative to a protein enzyme, in the fields related to medicine, pharmaceuticals, biochemistry and chemical engineering.

### Means for Solving the Problems

The inventors have diligently researched and found that a complex of a nucleotide, such as a nucleic acid, and a transition metal, such as platinum, shows catalytic activity.

Accordingly, the present invention provides a catalyst comprising a complex of a transition metal and a monomeric or polymeric nucleotide or an analogue thereof.

In an another aspect, the present invention also provides a peroxidase-like oxidation catalyst comprising a complex obtained by mixing in darkness a monomeric or polymeric nucleotide or an analogue thereof and a complex of a metal selected from the platinum group in an aqueous reaction medium selected from phosphate buffer solutions, borate buffer solutions and disodium hydrogenphosphate-sodium hydroxide buffer solutions under a neutral to alkaline condition.

### ADVANTAGES OF THE INVENTION

According to the present invention, it is possible to provide a catalyst (artificial enzyme) which can be used as an alternative to a protein enzyme (e.g., horseradish peroxidase: HRP), which generally has the disadvantage of susceptibility to denaturation and deactivation.

The catalyst of the present invention can be artificially synthesized in large quantities, and thus produced at a lower cost than a naturally-derived enzyme. In addition, it can be easily obtained as a highly pure product.

The catalyst of the present invention can be easily bound to another substance such as a protein and a nucleic acid, while maintaining its catalytic activity.

### BRIEF DESCRIPTION OF THE DRAWINGS

FIG. 1 shows a Matrix-Assisted Laser Desorption/Ionization (MALDI) spectrum of the product that was obtained by reacting 5-mer nucleotide (sequence AGAGA) and potassium tetrachloroplatinate (II), K₂[PtCl₄], under a condition of pH 9 at room temperature (25° C.) for 24 hours.

FIG. 2 shows a dependence on reaction time of catalytic activity of the nucleotide-platinum complexes (50 nmol/L) that were obtained in Example 2 by reacting a decamer of (A-G) ([AG]₁₀, total: 20 mers), wherein adenine (A) and guanine (G) are linked alternately, and potassium tetrachloroplatinate (II), K₂[PtCl₄], under conditions of pH 7, 9 and 11 at room temperature (25° C.) and for various time periods. The catalytic activity of the nucleotide-platinum complex was calculated on the basis that 5.47 units of enzymatic activity of horseradish peroxidase correspond to an absorbance of 1.

FIG. 3 shows a dependence on sequence of catalytic activity of the nucleotide-platinum complexes (50 nmol) that were obtained in Example 2 by reacting various icosamers, [AG]₁₀, [A]₁₀, [C]₁₀, [G]₁₀ and [T]₁₀, and potassium tetrachloroplatinate (II), K₂[PtCl₄], under conditions of pH 7, 9 and 11 at room temperature (25° C.) for 72 hours. The catalytic activity of the nucleotide-platinum complex was calculated on the basis that 5.47 units of enzymatic activity of horseradish peroxidase correspond to an absorbance of 1.

FIG. 4 shows catalytic activity of HRP (1,000 units/mg; 5 μmol/L) and the various DNA-platinum complexes (5 μmol/L) that were obtained in Example 3 by reacting potassium tetrachloroplatinate (II), K₂[PtCl₄], and single stranded (ss) or double stranded (ds) DNA (derived from salmon testes) under conditions of pH 7, 9 and 11 at room temperature (25° C.) for 72 hours. The catalytic activity of the DNA-platinum complex was calculated on the basis that 5.47 units of enzymatic activity of horseradish peroxidase correspond to an absorbance of 1.

FIG. 5 shows a dependence on strand length of catalytic activity of the nucleotide-platinum complexes (5 nmol/L) that were obtained in Example 4 by reacting monomers (adenosine monophosphate (AMP) and adenosine triphosphate (ATP)), DNA of adenine 10-mer, 15-mer and 20-mer, and single stranded salmon testes DNA, respectively, and potassium tetrachloroplatinate (II), K₂[PtCl₄], under a condition of pH 9 at room temperature (25° C.) for 24 hours. The catalytic activity of the nucleotide-platinum complex was calculated on the basis that 5.47 units of enzymatic activity of horseradish peroxidase correspond to an absorbance of 1.

FIG. 6 shows a dependence on concentration of catalytic activity of the DNA-platinum complexes that were prepared in Example 5 by reacting single stranded salmon testes DNA and potassium tetrachloroplatinate (II), K₂[PtCl₄], under a condition of pH 9 (indicated by open square in the figure) or pH 11 (indicated by closed circle in the figure) at room temperature (25° C.) for 72 hours. For comparison, the enzymatic activity of horseradish peroxidase (indicated by open circle in the figure) is also shown. The catalytic activity of the DNA-platinum complexes was calculated on the basis that 5.47 units of enzymatic activity of horseradish peroxidase correspond to an absorbance of 1.

FIG. 7 shows catalytic (enzymatic) activity of horseradish peroxidase (0.5 nmol/L, open circle in the figure) and the DNA-platinum complexes (0.5 nmol/L, closed circle in the figure) prepared in Example 6 after 30-min heat treatment at various temperatures. The DNA-platinum complexes were prepared by reacting single stranded salmon testes DNA and potassium tetrachloroplatinate, K₂[PtCl₄], under a condition of pH 9 at room temperature (25° C.) for 72 hours. The catalytic (enzymatic) activity is expressed as the percentage of catalytic (enzymatic) activity after heat-treatment at 20° C.

FIG. 8 shows catalytic activity of the DNA-platinum complexes that were prepared in Example 9 by reacting the synthetic oligodeoxynucleotides (A: the 29-mer 5′-TGAAGGCTTGAGTAAATTATTCCATCATAG-3′; B: the 30-mer 5′-[CT]₂₉T-3′) and cis-diaminedichloro platinum (II) at various temperatures and for various time periods. Two peroxidase substrates, TMB and ABTS, were used in A. Only TMB was used as the substrate in B.

FIG. 9 shows staining of the oligonucleotide-platinum complexes prepared in Example 10 with naphthol derivative/benzidine derivative+aqueous hydrogen peroxide. The oligonucleotide-platinum complexes were prepared by reacting the synthetic oligodeoxynucleotide (5′-AGAGAGA-3′) and the two platinum complexes, cis-diaminedichloro platinum PtCl₂(NH₃)₂ or potassium tetrachloroplatinate (II) K₂[PtCl₄], under a condition of pH 9.2 at 80° C. for 2.5 hours. Aliquots of the prepared oligonucleotide-platinum complex solution were spotted on a nitrocellulose membrane, which was then stained with the staining solution, washed with water and air-dried. Left: the stained image of oligonucleotide-platinum complex obtained with K₂[PtCl₄]; Right: the stained image of the oligonucleotide-platinum complex obtained with cis-diaminedichloro platinum. Upper: 2-μL spots; Lower: 4-μL spots.

FIG. 10 shows an image of luminescence induced by the reaction of luminol with DNA-platinum complex or amino linker-modified DNA (SCFb cDNA)-platinum complex. The DNA-platinum complex was prepared by reacting the synthetic oligodeoxynucleotide (5′-amino linker-(AG)₂₉G-3′) and cis-diaminedichloro platinum PtCl₂(NH₃)₂ at 80° C. for 2.5 hours, and the SCFb cDNA-platinum complex was prepared by reacting cis-diaminedichloro platinum PtCl₂(NH₃)₂ and a cDNA fragment of SCFb (stem cell factor precursor protein) at 80° C. for 2.5 hours, in Example 11. Aliquots of the DNA-platinum complexes were spotted on a nitrocellulose membrane, which was treated at 80° C. for 30 min, and then dipped in a luminol reagent containing luminol and hydrogen peroxide. Then, the nitrocellulose membrane was overlayed on and exposed to a Polaroid film for 1 min. Left: the DNA (SCFb cDNA)-platinum complex; Right: the amino linker-modified DNA (AG30)-platinum complex. Upper: 2-μL spots; Lower: 4-μL spots.

FIG. 11 shows the detection result by dot hybridization of DNA labeled with a DNA-platinum complex in Example 12. The DNA-platinum complex was prepared with amino linker-modified SCFb cDNA fragment, and then was conjugated to another amino linker-modified SCFb cDNA fragment. This conjugate was used as DNA-platinum labeled probe. The probe was contacted with a nylon membrane, on which 300 ng of each of the four cDNA fragments SCFb (upper left), MFAP4 (lower left), NOV (upper right), Delta1 (lower right), 300 ng had been immobilized. Then, the membrane was stained using a peroxidase staining kit. Only upper left spot of SCFb was stained heavily, indicating that the SCFb cDNA fragment labeled with DNA-platinum complex hybridizes with the complementary strands specifically.

FIG. 12 is a graph showing the results of sandwich ELISA experiments using two labeled antibodies conjugated with, as a label, the oligonucleotide-platinum complex that was prepared in Example 13 by reacting a synthetic oligonucleotide (5′-amino linker-(AG)₂₉G-3′) and cis-diaminedichloro platinum PtCl₂(NH₃)₂ under a condition of pH 9.2 at 80° C. for 2.5 hours. Closed circles: antibody A (anti-human IgE antibody conjugated with the oligonucleotide-platinum complex via glutaraldehyde). Closed squares: antibody B (anti-human IgE antibody conjugated with the DNA-Pt complex via an aldehyde group introduced by periodate oxidation of dextran). As control, the results of sandwich ELISA by a horseradish peroxidase-labeled antibody are also shown (open triangles). The ordinate axis represents optical absorbance due to that TMB (3,3′,5,5′-tetramethylbenzidine) substrate turns yellow by horseradish peroxidase.

### BEST MODE FOR CARRYING OUT THE INVENTION

The catalyst of the present invention is characterized in that it comprises a complex of a transition metal and a monomeric or polymeric nucleotide or an analogue thereof.

In the complex, at least one of the ligands is the monomeric or polymeric nucleotide or analogue thereof, and the remaining ligands are not limited to nucleotides.

If two or more nucleotides or analogues thereof are bound as ligands to the same transition metal (central metal), they may be separate monomeric or polymeric nucleotides, or different nucleotides in the same polymeric nucleotide.

In the case where the ligand is the polymeric nucleotide or analogue thereof, different nucleotides in the same polymeric nucleotide can be bound to different transition metals (central metals).

The activities exhibited by the catalyst of the present invention include, for example, catalytic activities exhibited by oxidative enzymes such as peroxidase, glucoseoxidase, catalase, uricase, riboxidase, amino acid oxidase; transfer enzymes such as hexokinase; hydrolysis enzyme such as protease, amylase, acylase, cellulase, chymotrypsin, collagenase, deoxyribonuclease, ribonuclease, lipase, protease, urease; and the like.

The catalyst of the present invention is preferably an oxidation catalyst, and more preferably it exhibits peroxidase-like oxidation catalytic activity. In the present invention, peroxidase-like oxidation catalytic activity is generally the activity which can catalyze the following reaction:

H₂O₂+substrate(in reduced form)->H₂O+product(in oxidized form)

Peroxidase-like catalytic activity can be determined as follows, for example: 3,3′,5,5′-tetramethylbenzidine (TMB), which can be a substrate for peroxidase, and hydrogen peroxide (H₂O₂) are added in darkness; after a given time period, a reaction stopping solution (such as 1 mol/L phosphoric acid solution) is added so as to stop the reaction; and the difference between optical absorbance at 450 nm and optical absorbance at 595 nm is measured on a microplate reader. Alternatively, the luminol reaction can be used more simply to detect peroxidase-like oxidation catalytic activity. Specifically, the presence of peroxidase-like oxidation catalytic ability can be confirmed by detection of chemiluminescence at around 460 nm (purple blue color) after addition of hydrogen peroxide and luminol under alkaline conditions.

The utilization of a standard curve (absorbance-dose curve) previously created with horseradish peroxidase (HRP) of a given activity (e.g., 1,000 units/mg) allows to convert measurements by the above described method using, for example, TMB and H₂O₂ to enzymatic activity of the HRP. The catalyst of the present invention can exhibit 1/100 (0.01) or more times, preferably 1/50 or more times, more preferably 1/20 or more times, more preferably 1/10 or more times the oxidation catalytic activity of HRP (activity: 1,000 units/mg) in an equimolar amount.

Transition metals which can be used in the catalyst include scandium (Sc), titanium (Ti), vanadium (V), chromium (Cr), manganese (Mn), iron (Fe), cobalt (Co), nickel (Ni), cupper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), yttrium (Yb), ruthenium (Lu), hafnium (Hf), tantalum (Ta), tungsten (W), rhenium (Re), osmium (Os), iridium (Ir), platinum (Pt), gold (Au), mercury (Hg), actinium (Ac), thorium (Th), protactinium (Pa), uranium (U), neptunium (Np), plutonium (Pu), americium (Am), curium (Cm), berkelium (Bk), californium (Cf), einsteinium (Es), fermium (Fm), mendelevium (Md), nobelium (No), and lawrencium (Lr).

The transition metal is preferably selected from the platinum group consisting of ruthenium, rhodium, palladium, osmium, iridium and platinum, and is more preferably platinum or palladium, and still more preferably platinum.

Platinum is bound to the nucleotide or analogue thereof in the form of, for example, —PtCl₂—, —PtCl₃, —PtCl(H₂O⁺)—, —Pt(H₂O⁺)₂—, —PtCl₂(H₂O⁺), —PtCl(H₂O⁺)₂, —Pt(H₂O⁺)₃, —PtCl(NH₃)₂, —Pt(NH₃)₂— or —Pt(H₂O⁺)(NH₃)₂ in the complex.

The nucleotide in the catalyst may be a ribonucleotide or a deoxyribonucleotide, although a deoxyribonucleotide is preferable.

The representative examples of monomeric nucleotide include adenosine monophosphate (AMP), adenosine diphosphate (ADP), adenosine triphosphate (ATP), thymine monophosphate (TMP), thymine diphosphate (TDP), thymine triphosphate (TTP), cytosine monophosphate (CMP), cytosine diphosphate (CDP), cytosine triphosphate (CTP), guanosine monophosphate (GMP), guanosine diphosphate (GDP), guanosine triphosphate (GTP), deoxyadenosine monophosphate (dAMP), deoxyadenosine diphosphate (dADP), deoxyadenosine triphosphate (dATP), uridine monophosphate (UMP), uridine diphosphate (UDP), uridine triphosphate (UTP), deoxycytosine monophosphate (dCMP), deoxycytosine diphosphate (dCDP), deoxycytosine triphosphate (dCTP), deoxyguanosine monophosphate (dGMP), deoxyguanosine diphosphate (dGDP), and deoxyguanosine triphosphate (dGTP).

The polymeric nucleotide is a polymer of two or more monomeric nucleotides. Indeed, the catalyst wherein the nucleotide is a dimer exhibit enzymatic activity. However, as the number of nucleotides in the polymeric nucleotide increases, the number of transition metals bindable to a molecule of the polymeric nucleotide (i.e., the active sites of such a catalyst) increases, and therefore higher catalytic activity can be obtained. Thus, the polymeric nucleotide in the catalyst has preferably at least 5 consecutive nucleotides, more preferably at least 7 consecutive nucleotides, more preferably at least 10 consecutive nucleotides, more preferably at least 15 consecutive nucleotides, more preferably at least 20 consecutive nucleotides, and more preferably at least 30 consecutive nucleotides.

The upper limit of the number of nucleotides in the polymeric nucleotide is not specified, and can be 100,000 or less, for example.

The polymeric nucleotide may be single stranded, double stranded, cyclic or branched (e.g., dendrimer), and preferably single stranded.

The polymeric nucleotide may be a nucleic acid, preferably a single stranded nucleic acid. The nucleic acid may be a deoxyribonucleic acid (DNA) or a ribonucleic acid (RNA). Because phosphodiester bond in a nucleic acid (especially DNA) is 100 or more times stable against hydrolysis than a peptide bond in a protein, the catalyst comprising a complex of a nucleic acid (especially DNA) and a transition metal is more chemically stable than a protein enzyme, and it is resistant to deactivation in the course of a long-term storage. Such a catalyst can also maintain its catalytic activity even at high temperature. DNA is especially preferable because it is more stable than even RNA.

The DNA can be any DNA. A DNA of biological origin may be used in itself, or a fragment thereof may be used which is obtained by digestion with a restriction enzyme. Alternatively, an artificially synthesized DNA (e.g., using a DNA synthesizer) may be used.

The DNA of biological origin include, for example, DNA from calf thymus, DNA from salmon testes, single stranded DNA from salmon testes, DNA from E. Coli, lambda phage DNA, DNA from human cells, and the like. The DNA of biological origin can be obtained by extraction or the like from a biological tissue. A commercially available DNA may be used.

In the present invention, DNA having 1 to 100,000 bases can be used.

DNA can be used, which has a specially designed base sequence, such as a base sequence where adenine is repeated at the base moieties, (A)ₙ; a base sequence where thymine is repeated at the base moieties, (T)ₙ; a base sequence where cytosine is repeated at the base moieties, (C)ₙ; a base sequence where guanine is repeated at the base moieties, (G)ₙ; a base sequence where adenine and guanine are repeated at the base moieties, (AG)ₙ; a base sequence where cytosine and thymine are repeated at the base moieties, (CT)ₙ; and a combination thereof (where n is a repeated unit number, and is usually an integer from 1 to 300).

The above DNA can be condensed with a DNA ligase enzyme. An elongated DNA which may be used which has 100 to 100,000 bases, or a DNA may be used which has a branched structure such as a dendrimer structure.

Large amounts of DNA having the same sequence can be easily produced by amplification of a template DNA using PCR (polymerase chain reaction) methods.

The RNA can be any RNA. An RNA of biological origin may be used in itself, or a fragment thereof may be used which is obtained by digestion with a restriction enzyme. Alternatively, an artificially synthesized RNA (e.g., using an RNA synthesizer) may be used.

The RNA of biological origin includes, for example, RNA from yeast, transfer RNA from yeast, and the like. The RNA of biological origin can be obtained by extraction or the like from a biological tissue. A commercially available DNA may be used.

In the present invention, RNA having 1 to 100,000 bases can be used.

RNA can be used, which has a specifically designed base sequence, such as a base sequence where adenine is repeated at the base moieties, (A)ₙ; a base sequence where uracil is repeated at the base moieties, (U)ₙ; a base sequence where cytosine is repeated at the base moieties, (C)ₙ; a base sequence where guanine is repeated at the base moieties, (G)ₙ; a base sequence where adenine and guanine are repeated at the base moieties, (AG)ₙ; a base sequence where cytosine and uracil are repeated at the base moieties, (CU)ₙ; and a combination thereof (where n is a repeated unit number, and is usually an integer from 1 to 300).

The above DNA can be further condensed with an RNA ligase enzyme. An elongated RNA may be used which has 100 to 100,000 bases, or a DNA may be used which has a branched structure such as a dendrimer structure.

The above monomeric or polymeric nucleotide may be an artificially modified/substituted nucleotide analogue. Analogues of the polymeric nucleotide are those polymeric nucleotides wherein one or more sugar moieties and/or internucleotide bonds and/or base moieties are modified. The sugar moiety can be replaced with, for example, 3′-deoxyribocyl, 2′,3′-dideoxyribocyl, 2′,3′-didehydrodideoxycyribocyl, 2′- or 3′-alcoxyribocyl, 2′- or 3′-azidoriboxyl, 2′- or 3′-aminoribocyl, 2′- or 3′-fluororibocyl, 2′- or 3′-mercaptoribocyl, 2′- or 3′-alkylthioribocyl, or any other modified ribocyl. The internucleotide bond can be replaced with, for example, phosphorothioate, phosphorodithioate, phosphoroamidate, phosphoroserenoate, phosphorodiserenoate, phosphoroanilothioate, or any other phosphodiester analogue. The analogue can contain, as the base moiety, adenine, guanine, cytosine, thymine and uracil, as well as, inosine, hypoxanthine, xanthine, a methylated base, modified base in tRNA, or the like. It also can contain a base modified with an amino group, an SH group, a biotin group, a phosphate group, a saccharide group, a fluorescent dye (e.g., fluorescein or Cy3), or the like.

The above catalyst of the present invention can be produced by a method comprising reacting in darkness a monomeric or polymeric nucleotide or an analogue thereof as described above and a transition metal complex as described above in an aqueous reaction medium under a neutral to alkaline condition and collecting the reaction product.

As the transition metal complex, any complexes of transition metals as mentioned above can be used, although preferable is a complex of a metal selected from the platinum group. A platinum complex is especially preferable in view of stability and chemical reactivity.

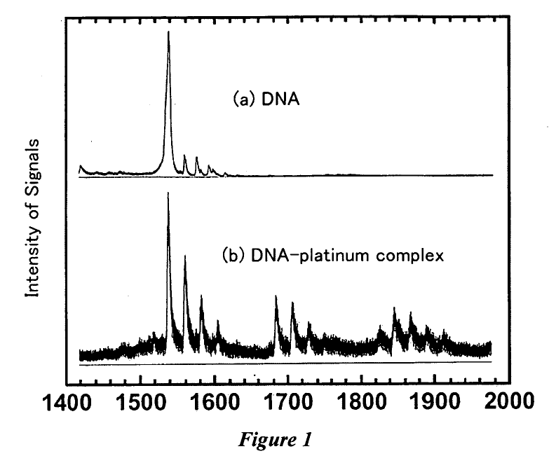

A platinum complex can be used which has at least one ligand that is capable of reacting with the nucleotide or its analogue. Also, a platinum complex can be used which has C₁, H₂O, NO₃, CN, N₃, (CH₃)₂SO, PO₄, CO₃ or the like as a leaving ligand that is capable of reacting with the nucleotide or its analogue by ligand exchange reaction. Such platinum complexes include, for example, potassium tetrachloroplatinate (II) (K₂[PtCl₄]), cis-dichlorodiamine platinum (II) (CDDP: cisplatin), puriplatin, carboplatin (CBDCA), paraplatin, aqupra (Nedaplatin; 254-S), trans-dichlorodiamine platinum, tetrachlorodiamine platinum, ibroplatin, maronatoplatinium, DACCP, Pt(dien)Cl, dichloro(N-ethylethylenediamine) platinum (II) (see, FIG. 1 of non-patent reference 11), dichloro(N-propylethylenediamine) platinum (II) (see, FIG. 1 of non-patent reference 11), and the like.

The neutral to alkaline condition is pH 7 to 14, preferably pH 7 to 11.

The aqueous reaction medium can be any aqueous reaction medium that does not interfere with the reaction between the nucleotide or its analogue and the transition metal complex. For example, it can be selected from phosphate buffer solutions, borate buffer solutions and disodium hydrogenphosphate-sodium hydroxide buffer solutions.

The nucleotide, for example, prepared in pure water and the transition metal complex, for example, prepared in pure water are mixed and reacted in darkness (e.g., in a light-resistant container) in an aqueous reaction medium as described above under a neutral-alkaline condition.

The reaction temperature is not limited specifically, as long as it is a temperature at which the reaction of the nucleotide and the transition metal complex occurs in the aqueous reaction medium, although it is usually room temperature (25° C.) to 100° C. The reaction temperature is desirable where the transition metal complex does not decompose.

The reaction time depends on the reaction temperature. In the case where the reaction temperature is room temperature (e.g., where the transition metal complex is potassium tetrachloroplatinate (II)), the reaction time is desirable to be 1 hour or more. If the reaction temperature is set to be higher, the reaction time can be set shorter. For example, where the reaction temperature is, for example, 80 to 98° C. (e.g., where the transition metal complex is cis-dichlorodiamine platinum (II)), the reaction time can be 10 min or more, for example.

Specific examples of the reaction temperature and time are 25 to 37° C. for 1 hour to 12 days, preferably 24 hours to 120 hours (such as 24 hours, 72 hours, 120 hours); and 80 to 95° C. for 10 min to 3 hours, preferably 30 min to 3 hours, more preferably 1 to 3 hours.

The formed complex of the nucleotide and the transition metal can be collected by, for example, an ethanol-precipitation method, a centrifuged filtration method, or a gel filtration method. An exemplified ethanol precipitation method comprises: adding 1/10 volume of 3 mol/L sodium acetate and 2.5 volumes of ethanol to one volume of the reaction solution, and stirring it; and then letting it stand at −20° C. for 30 min to 12 hours, to allow for precipitation; separating the precipitates by centrifugation at room temperature at 15,000 rpm for 15 minutes; adding 2.5 volumes of 70% ethanol to the obtained precipitates; separating again the precipitates by centrifugation for 15 min at 15,000 rpm; and drying the obtained precipitates.

The binding between the nucleotide (e.g., DNA or RNA) and the transition metal can be confirmed by an increase in molecular weight of the nucleotide. The measurement of molecular weight can be performed with mass spectroscopy using a MALDI method or an ion spray ionization method. Furthermore, the binding between the nucleotide and the transition metal can be confirmed by determining the binding state with Nuclear Magnetic Resonance spectrum, X-ray structural analysis, and X-ray photoelectron spectrometer (XPS).

The complex of the nucleotide or its analogue and the transition metal complex as produced by the above method exhibits such catalytic activity that is not exhibited by the nucleotide alone, the transition metal complex alone, which is used in the production, or the transition metal complex's ligand alone.

The above catalyst of the present invention can be used as an alternative to a protein enzyme (such as peroxidase or alkaline phosphatase) in a catalytic reaction with such an enzyme.

The above catalyst of the present invention also can be used as an alternative to a protein enzyme in a conventional detection or quantification method, and a reagent therefor, utilizing such an enzyme.

For example, the catalyst of the present invention can be associated, directly or via a linker, with a substance capable specifically binding a target substance for detection, so that it is used as a labeled reagent for detection or quantification of the target substance.

The substance capable of specifically binding the target substance is, for example, a protein having molecular recognition ability, such as an antibody (such as an IgG antibody, an IgM antibody, or the like), avidin, protein A, protein G, lectin or the like; or a nucleotide probe (nucleic acid probe).

For direct binding of the catalyst of the present invention to a protein, for example, glutaraldehyde or water-soluble carbodiimide (1-ethyl-3-(3-dimethylaminopropyl) carbodiimide hydrochloride) is used.

The catalyst of the present invention can be bound to a protein through the reaction of the protein with a maleimide derivative, or via intermediate substance such as gold colloid, dextran, polyethyleneglycol, ceramic beads or plastic beads.

For direct binding of the catalyst of the present invention with nucleic acid probe, for example, DNA ligase (in the case of double stranded DNA), RNA ligase (in the case of single stranded DNA and RNA), or water-soluble carbodiimide (1-ethyl-3-(3-dimethylaminopropyl) carbodiimide hydrochloride) is used. The catalyst of the present invention wherein an SH group has been introduced, may be bound to an SH group created on the nucleic acid probe using maleimidebenzoyl-N-hydroxysuccinimide ester or a similar bifunctional reagent.

A functional group (such as an amino group, an SH group, a biotin group, a phosphate group and saccharide group) that have been introduced in the nucleotide moiety of the catalyst of the present invention, may be used for binding the catalyst with a protein or a nucleic acid. The introduction of a functional group as described above in the nucleotide moiety of the catalyst of the present invention can be performed by using a synthetic nucleotide wherein such a functional group have been previously introduced during synthesis of the nucleotide moiety.

The above reagent can be included, in place of a reagent using a protein enzyme, in a conventional kit for detection or quantification of a target substance for detection.

The above reagent can be used for detecting the presence of a target substance for detection in a sample, by contacting the sample with the reagent, and determining the presence/absence of the catalytic activity after removing any untreated reagent.

The above reagent also can be used for quantifying an amount of a target substance for detection in a sample, by contacting the sample with the reagent, measuring the catalytic activity after removing any unreacted reagent, and comparing the measured value with a predetermined standard curve.

The above reagent is used, in the detection or quantification method, in combination with ELISA (enzyme-linked immunosorbent assay) method, immunoprecipitation method, immunochromatography method, western blotting method, histochemical detection method, Southern blotting hybridization method, Northern blotting hybridization method, colony hybridization method, plaque hybridization method, dot hybridization method, DNA chip method, gene chip method, or DNA microarray method.

In another aspect, the present invention relates to a use of a monomeric or polymeric nucleotide or an analogue thereof associated with a complex of a transition metal, as an oxidation catalyst.

The use of the present invention is preferable also in a high temperature oxidation reaction conducted at 50° C. or more.

The modes of the use as an oxidation catalyst include, for example, the use as a label utilizing oxidation catalytic reaction for detection/quantification, and the use in the preparation of a reagent for detection or quantification

### EXAMPLES

Hereinafter, the present invention will be described by means of the following examples, although it is not limited to the examples.

### Example 1

TE buffer was prepared according to the following procedures; 1.21 g (10 mmol) of Tris(hydroxymethyl)aminomethane (121.14 g/mol) and 0.37 g (1 mmol) of ethylenediamine tetraacetic acid disodium dihydrate (372.2 g/mol) were dissolved in 1 L of pure water. This solution was sterilized in an autoclave for one hour, and was subsequently cooled to room temperature. The pH of the TE buffer was adjusted to be 8.0 with 0.1 M NaOH or 0.1 M HCl aqueous solution.

A single stranded DNA originated from salmon testes (D-7656, molecular weight 468,000, purchased from Sigma-Aldrich Japan, Ltd.) or 5-mer DNA having an alternately repeated sequence of adenine [A] and guanine [G], AGAGA, was dissolved in pure water to prepare 1000 ppm DNA solutions.

Potassium tetrachloroplatinate (II) (K₂[PtCl₄]=415.9 g/mol, N.E. Chemical, Inc.) (116.62 mg) was put into an Eppendorf tube shaded with aluminum foil, and dissolved in pure water to prepare 1.4 mL of a platinum complex solution (83,300 ppm).

Seventy μL of a phosphate buffer solution (pH 7.0), 5 μL of the platinum complex solution, and 25 μL of the DNA solution were put into a 1.5-mL Eppendorf tube, which was in turn shaded with aluminum foil, and were subsequently reacted at pH 7.0 at room temperature (25° C.) for 24 hours to prepare a complexed compound of DNA with platinum complex. Another complexed compound of DNA with platinum complex by the reaction for 120 hours was also prepared similarly.

Furthermore, for the preparation of another platinum complex-DNA by a reaction at pH 9.2, 70 μL of a borate buffer solution (pH 9.2, Wako Pure Chemicals Ltd., 028-03205), 5 μL of the platinum complex solution, and 25 μL of the DNA solution were put into a 1.5-mL Eppendorf tube, which was in turn shaded with aluminum foil, and were subsequently reacted at pH 9.2 at room temperature (25° C.) for 24 hours to prepare the complexed compound of DNA with platinum complex. Another complexed compound of DNA with platinum complex by the reaction for 120 hours was also prepared similarly.

For the preparation of another complexed compound of DNA with platinum complex by a reaction at pH 11.0, 70 μL of disodium hydrogenphosphate-sodium hydroxide buffer solution (pH 11.0), 5 μL of the platinum complex solution, and 25 μL of the DNA solution were put into a 1.5-mL Eppendorf tube, which was in turn shaded with aluminum foil, and were subsequently reacted at pH 11.0 at room temperature (25° C.) for 24 hours to prepare to the complexed compound of DNA with platinum complex. Another complexed compound of DNA with platinum complex by the reaction for 120 hours was also prepared similarly.

To the above-prepared solution of the complexed compound of DNA with platinum complex, one-tenth times its volume of 30 mol/L sodium acetate and 2.5 times its volume of ethanol were added and stirred well. This solution was kept at −20° C. in a refrigerator for 12 hours to age the precipitates. And then, the complexed compound of DNA with platinum complex was further precipitated with a centrifuge (at 15,000 rpm and room temperature for 15 min). After removing the supernatant, 70% ethanol solution was further added in an amount of 2.5 times the volume of the solution of the complexed compound of DNA with platinum complex, and another centrifugation was performed (at 15,000 rpm and room temperature for 15 min). After removing the supernatant, the precipitate was dried to obtain a purified, complexed compound of DNA with platinum complex.

Elementary analyses of the complexed compounds of DNA with platinum complex that had been prepared using the single stranded salmon testes DNA, were performed by an X-ray photoelectron spectroscopy. Table 1 shows the results of the complexed compounds of DNA with platinum complex, which were prepared under conditions of pH 7, 9 or 11 for 24 or 120 hours. If the platinum complexes are bound to all of the base residues of the DNA, the atomic ratio of platinum/phosphorus is 1, because a monomer of DNA contains one phosphorus. It was calculated from Table 1 that on average, one platinum complex is bound to 1.7 of 20-mer DNA, in the case of the complexed compound of DNA with platinum complex that was prepared at pH 11 for 120 hours. The atomic ratio of platinum/phosphorus increases with an increase in reaction time. From this, it is evident that the formation rate of the complexed compound of DNA with platinum complex increases with an increase in reaction time. When the reaction time is the same, the atomic ratio of platinum/phosphorus increases with an increase in pH. From this, it is also evident that the formation rate of the complexed compound of DNA with platinum complex increases with an increase in pH of the reaction

TABLE 1
Atomic ratios, measured by XPS, of DNA-platinum
complexes prepared under various conditions

| | | Reaction | Atomic ratio | Atomic ratio |
|---|---|---|---|---|
| | Reaction | time | (platinum/ | (chloride/ |
| Sample | pH | (hour) | phosphorus) | platinum) |
| DNA-platinum | 7 | 24 | 7.19 × 10⁻³ | 3.57 |
| complex No. 1 | | | | |
| DNA-platinum | 11 | 24 | 21.9 × 10⁻³ | 1.33 |
| complex No. 2 | | | | |
| DNA-platinum | 7 | 120 | 7.96 × 10⁻³ | 2.75 |
| complex No. 3 | | | | |
| DNA-platinum | 9 | 120 | 17.7 × 10⁻³ | 0.61 |
| complex No. 4 | | | | |
| DNA-platinum | 11 | 120 | 29.5 × 10⁻³ | 0.47 |
| complex No. 5 | | | | |

A complexed compound of DNA with platinum complex was prepared similarly to Example 1 except that 250 ppm of the 5-mer DNA (which has a sequence wherein adenine [A] and guanine [G] are linked, AGAGA) and 250 ppm of platinum complex (potassium tetrachloroplatinate (II)) solution were used and reacted at pH 7 for 24 hours. The complexed compound of DNA with platinum complex was analyzed by using a Matrix-Assisted Laser Desorption/Ionization (MALDI) method to confirm that the complexed compound actually forms a complex. The result is shown in FIG. 1. In addition to a peak due to the molecular weight (1536 daltons) of the 5-mer DNA, peaks were observed at the positions corresponding to the molecular weight of the 5-mer DNA plus the molecular weight of one molecule of the platinum complex (from 1800 (—PtCl₂— binding) to 1840 (—PtCl₃ binding)). The peaks observed at positions corresponding to the other molecular weights in FIG. 1 are considered to be due to the substances obtained by binding ions to the complexed compound of DNA with platinum complex. It is evident from the above results that there exists a complexed compound of DNA with platinum complex where at least one molecule of the platinum complex is bound to the DNA.

### Example 2

Purified complexed compounds of DNA with platinum complex were prepared similarly to Example 1 except that 20-mer DNAs (adenine 20-mer (A)₂₀, guanine 20-mer (G)₂₀, cytosine 20-mer (C)₂₀, thymine 20-mer (T)₂₀, adenine+guanine 10-mer (AG)₁₀, total 20-mer DNAs, purchased from Sigma-Aldrich Japan, Ltd., a product purified through a cartridge, 1.0 μmol each) were used.

In order to evaluate the catalytic activity of the prepared, complexed compounds of DNA with platinum complex, the peroxide enzymatic activity (peroxidase activity) was measured as follows.

The Purified complexed compounds of DNA with platinum complex each were dissolved into 0.2 mL of the TE buffer solution (pH 8.0). The solutions of the complexed compound of DNA with platinum complex were diluted with pure water to be 50 nmol/L on the basis of DNA concentration, and 0.18 mL each of the solutions was put into each well of a 24-well plate. Equal amounts of a solution of the peroxidative enzyme substrate 3,3′,5,5′-tetramethylbenzidine (TMB peroxidative enzyme substrate solution, Funakoshi Co. Ltd., 50-76-01) and a hydrogen peroxide solution (0.02%, peroxidative enzyme substrate solution B, Funakoshi Co. Ltd., 50-76-00) were mixed, and 0.18 mL of the mixture solution was put into each well of the 24-well plate. The plate was shaded with aluminum foil and placed on a micro vibrator for 20 min. Then, in the mixed solution in the 24-well plate after the TMB reaction, 0.18 mL per well of 1 mol/L phosphoric acid solution (Wako Pure Chemicals Ltd.; special reagent grade) as a TMB reaction stop reagent is added to stop the reaction.

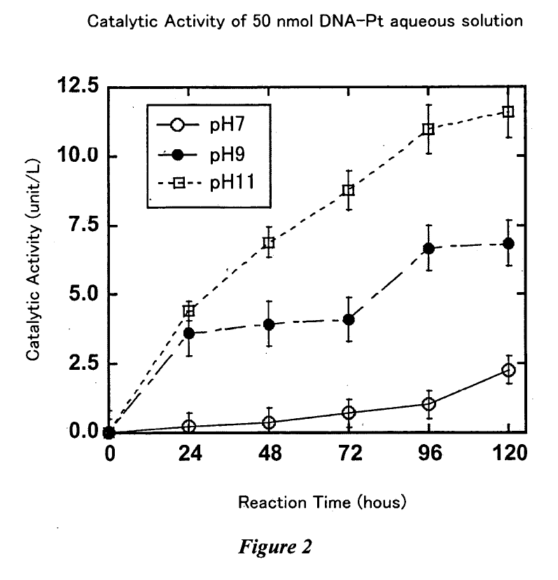

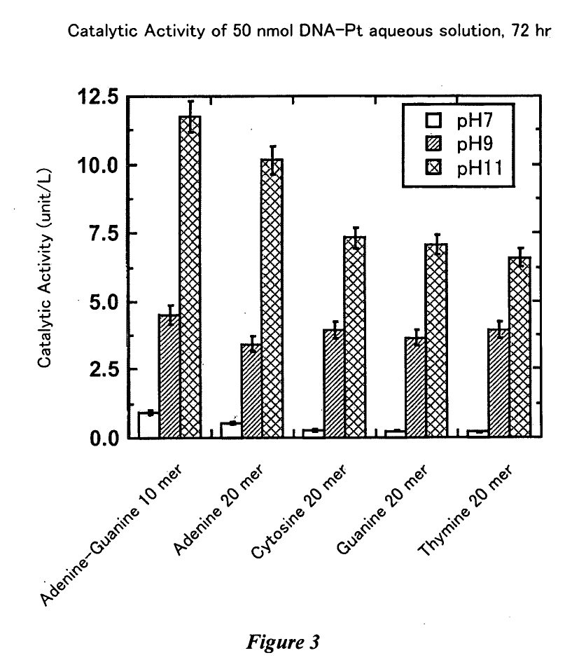

Each 0.2 mL of the reaction solutions was put into each well of a 96-well plate, and then the absorbance difference of the solutions at 450 and 595 nm was measured using a microplate reader (Bio-Rad Model 550). The enzymatic (catalytic) activity of the horseradish peroxidase was measured following the same procedure described above but using 0.5 nmol/L of the horseradish peroxidase (1000 units/mg, D-7656, molecular weight 40,000, purchased from Sigma-Aldrich Japan, Ltd.). By comparing the ratio of the absorbance increase due to the reaction of the substrate with the horseradish peroxidase and that due to the reaction of the substrate and the complexed compounds of DNA with platinum complex, the reactivity of the complexed compounds of DNA with platinum complex was compared with that of the horseradish peroxidase (in this Example, it is calculated that an absorbance of 1 corresponds to 5.47 units). The reactivity is shown in FIGS. 2 and 3.

It is evident from FIG. 2 that enzymatic (catalytic) activity of the complexed compounds of DNA with platinum complex increases with an increase in reaction time. It is evident from FIG. 3 that the enzymatic activity found with the DNA having any of the base sequences. As for the pH in the preparation of the complexed compounds of DNA with platinum complex, it is evident that the complexed compounds of DNA with platinum complex that were prepared at higher pH of the reaction solution, have higher enzymatic (catalytic) activity. It is also observed that the enzymatic (catalytic) activity of the complexed compounds of DNA with platinum complex depends, to some extent, on the base sequence of the DNA used.

From the above, it is evident that the complexed compounds of DNA with platinum complex have a peroxidation enzyme activity.

### Comparative Example 1

Peroxidation enzyme (peroxidase) activity was measured, as below, similarly to Example 2 except using 20-mer DNAs (adenine [A] 20-mer, guanine [G] 20-mer, cytosine [C] 20-mer, thymine [T] 20-mer, and [A-G] 10-mer, (AG)₁₀, where adenine [A] and guanine [G] were linked, total 20 mers, purchased from Sigma-Aldrich Japan, Ltd., a product purified through a cartridge, 1.0 μmol each) instead of the complexed compounds of DNA with platinum complex.

The above DNAs each were dissolved into 0.2 mL of the TE buffer solution (pH 8.0). The DNA solutions were diluted with pure water to be 50 nmol/L on the basis of DNA concentration, and 0.18 mL each of the solutions was put into each well of a 24-well plate. Equal amounts of a solution of the peroxidative enzyme substrate 3,3′,5,5′-tetramethylbenzidine (TMB peroxidative enzyme substrate solution A, Funakoshi Co. Ltd., 50-76-01) and a hydrogen peroxide solution (0.02%, peroxidative enzyme substrate solution B, Funakoshi Co. Ltd., 50-76-00) were mixed, and 0.18 mL of the mixture solution was put into each well of the 24-well plate. The plate was shaded with aluminum foil and placed on a micro vibrator for 20 min. Then, in the mixed solution in the 24-well plate after the TMB reaction, 0.18 mL per well of 1 mol/L phosphoric acid solution (Wako Pure Chemicals Ltd.; special reagent grade) as a TMB reaction stop reagent is added to stop the reaction.

Each 0.2 mL of the reaction solutions was put into each well of a 96-well plate, and then the absorbance difference of the solutions at 450 and 595 nm was measured using a microplate reader (Bio-Rad Model 550). The absorbance difference of any of the DNA solutions was zero within the experimental error. It is evident that DNAs alone does not have the peroxidative enzyme (peroxidase) activity, which the complexed compound of DNA with platinum complex have.

### Comparative Example 2

Peroxidative enzyme (peroxidase) activity was measured, as below, similarly to Example 2 except using potassium tetrachloroplatinate (II) (K₂[PtCl₄]=415.9 g/mol, N.E. Chemical Co., Ltd.) instead of the complexed compounds of DNA with platinum complex.

The potassium tetrachloroplatinate (II) was dissolved into 0.2 mL of the TE buffer solution (pH 8.0) to obtain 50 nmol/L solution. Then, 0.18 mL of the 50 nmol/L solution was put into each well of a 24-well plate. Equal amounts of a solution of the peroxidative enzyme substrate 3,3′,5,5′-tetramethylbenzidine (TMB peroxidative enzyme substrate solution A, Funakoshi Co. Ltd., 50-76-01) and a hydrogen peroxide solution (0.02%, peroxidative enzyme substrate solution B, Funakoshi Co. Ltd., 50-76-00) were mixed, and 0.18 mL of the mixture solution was put into each well of the 24-well plate. The plate was shaded with aluminum foil and placed on a micro vibrator for 20 min. Then, in the mixed solution in the 24-well plate after the TMB reaction, 0.18 mL per well of 1 mol/L phosphoric acid solution (Wako Pure Chemicals Ltd.; special reagent grade) as a TMB reaction stop reagent is added to stop the reaction.

Each 0.2 mL of the reaction solutions was put into each well of a 96-well plate, and then the absorbance difference of the solutions at 450 nm and 595 nm was measured by using a microplate reader (Bio-Rad Model 550). The absorbance difference of the potassium tetrachloroplatinate (II) solution at 450 nm and 595 nm was zero within the experimental error. It is evident that the platinum complex alone does not have the peroxidation enzyme (peroxidase) activity, which the complexed compounds of DNA with platinum complex do. Also, chloride ion and ammonium ion, ligands of the platinum complex, do not have such activity.

### Example 3

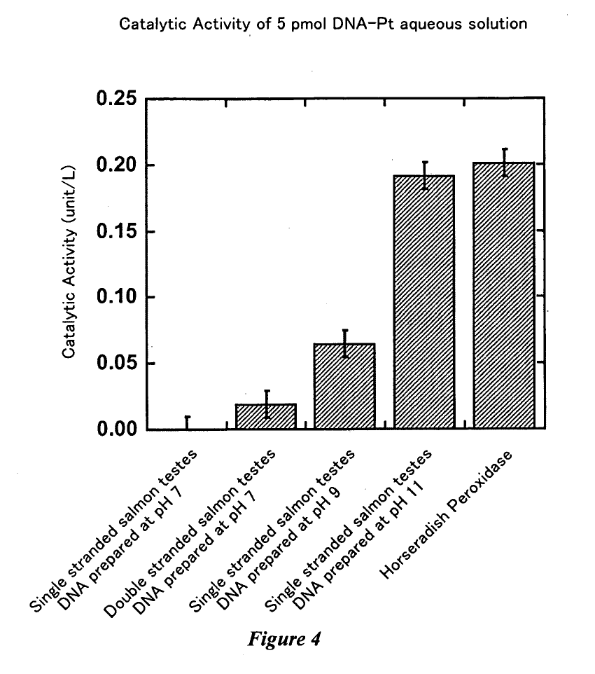

Purified complexed compounds of DNA with platinum complex were prepared similarly to Example 1 except using single stranded salmon testes DNA (D-7656, molecular weight 468,000, purchased from Sigma-Aldrich Japan, Ltd.) or double stranded salmon testes DNA (D-1626, purchased from Sigma-Aldrich Japan, Ltd.), and a reaction time of 72 hours. Then, the enzymatic (catalytic) activity of the purified, complexed compounds of DNA with platinum complex and the horseradish peroxidase was measured similarly to Example 2 except using 5 μmol of the complexed compounds of DNA with platinum complex or 5 μmol of the horseradish peroxidase (1000 units/mg, D-7656, molecular weight 40,000, purchased from Sigma-Aldrich Japan, Ltd.). The results are shown in FIG. 4. It is evident that enzymatic activity is also exhibited by complexed compounds of DNA with platinum complex that were prepared using a salmon testes DNA with a high molecular weight. It is also evident that the complexed compound of DNA with platinum complex that was prepared using the single stranded DNA, is higher in the enzymatic activity than the complexed compound of DNA with platinum complex that was prepared using the double stranded DNA (the molecular weight of which is shown as 936,000 in FIG. 4). It is also evident that the complexed compound of DNA with platinum complex that is prepared at pH 11 exhibits the enzymatic (catalytic) activity comparably to a commercially available enzyme (horseradish peroxidase).

### Example 4

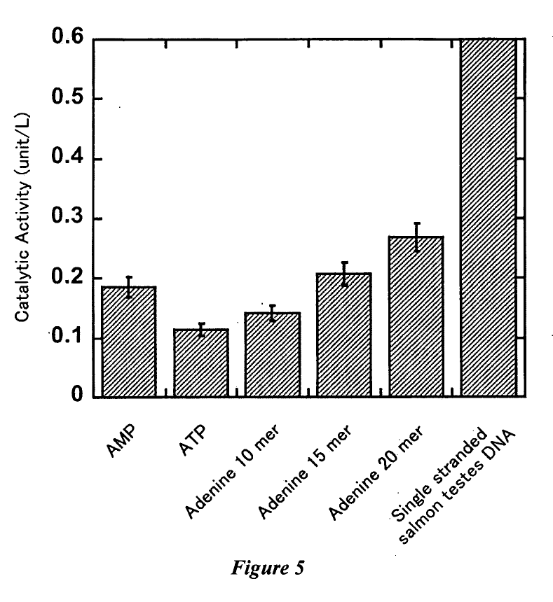

Purified complexed compounds of DNA with platinum complex were prepared, similarly to Example 1, using a monomer nucleotide (adenosine monophosphate (AMP), adenosine triphosphate (ATP)), adenine 10-mer, adenine 15-mer, adenine 20-mer (adenine 10-mer, 15-mer and 20-mer were purchased from Sigma-Aldrich Japan, Ltd., a product purified through a cartridge, 1.0 μmol each), or single stranded salmon testes DNA (D-7656, molecular weight 468,000, purchased from Sigma-Aldrich Japan, Ltd.) under a condition of pH 9 for a reaction time of 24 hours. The enzymatic (catalytic) activity of the purified, complexed compounds of DNA with platinum complex was measured similarly to Example 2 except using 5 nmol/L of the complexed compounds of DNA with platinum complex. The results are shown in FIG. 5. It is evident that the complexed compounds of DNA with platinum complex that were prepared using ATP or AMP exhibits the enzymatic activity comparably to those prepared from 10- to 15-mer DNAs. This may be because ATP and AMP are monomers and thus have high reactivity to the platinum complex. It is also evident that in the complexed compounds of DNA with platinum complex that were prepared using 10-mer or more DNAs, the enzymatic (catalytic) activity per unit strand of the molecule increases with an increase in the strand length of the DNAs. In the complexed compounds of DNA with platinum complex that were prepared using adenine 10-mer, 15-mer and 20-mer, the enzymatic (catalytic) activity per one residue of the DNA was calculated as 0.014, 0.014 and 0.013 unit/L, respectively. It is evident that purified, complexed compounds of DNA with platinum complex that were prepared at pH 9 for a reaction time of 24 hours, exhibits 0.014±0.001 units/L of enzymatic (catalytic) activity.

Similar experiments were performed using adenine 2-mer and 5-mer (both are synthesized from Sigma-Aldrich custom order, a product purified with a cartridge). The one prepared using the 5-mer exhibits about one half of the activity of the one prepared using adenine 10-mer. The one prepared using 2-mer exhibits the activity comparably to the one prepared using ATP.

### Example 5

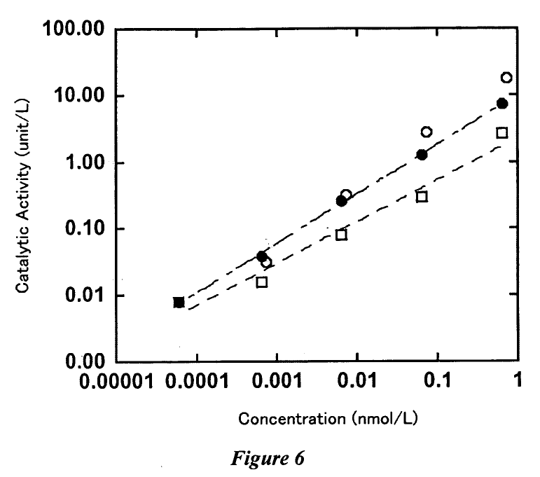

Purified, complexed compounds of DNA with platinum complex were prepared, similarly to Example 1, using single stranded salmon testes DNA (D-7656, molecular weight 468,000, purchased from Sigma-Aldrich Japan, Ltd.) under a condition of pH 9 or 11 for a reaction time of 72 hours. The enzymatic activity of the purified, complexed compounds of DNA with platinum complex and the horseradish peroxidase was measured similarly to Example 2 except using the complexed compounds of DNA with platinum complex and the horseradish peroxidase (1000 units/mg, D-7656, molecular weight=40,000, purchased from Sigma-Aldrich Japan, Ltd.). The results are shown in FIG. 6. It is evident that in any of the cases, the enzymatic activity increases with an increase in concentration of the complexed compounds of DNA with platinum complex or horseradish peroxidase. It is also evident that, in particular, the complexed compound of DNA with platinum complex that was prepared at pH 11 for a reaction time of 72 hours, exhibits the enzymatic (catalytic) activity equivalent to the horseradish peroxidase. The enzymatic (catalytic) activity of the complexed compounds of DNA with platinum complex increases with an increase in concentration, like the horseradish peroxidase. From this, it is evident that in a medical diagnostic kit based on enzyme-immunoassay wherein horseradish peroxidase is used, the complexed compounds of DNA with platinum complex can be used instead of said horseradish peroxidase.

### Example 6

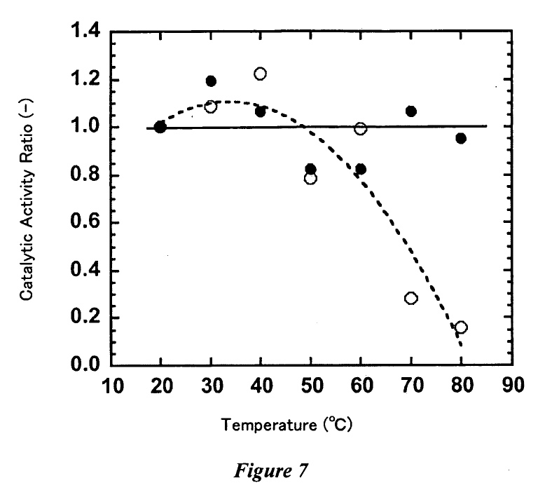

Purified, complexed compounds of DNA with platinum complex were prepared, similarly to Example 1, using single stranded salmon testes DNA (D-7656, molecular weight 468,000, purchased from Sigma-Aldrich Japan, Ltd.) under a condition of pH 9 for a reaction time of 72 hours. In an isotherm bath, 0.5 nmol/L solution of the purified, complexed compounds of DNA with platinum complex and 0.5 nmol/L horseradish peroxidase solution (1000 units/mg, D-7656, molecular weight 40,000, purchased from Sigma-Aldrich Japan, Ltd.) were heated at 20, 30, 40, 50, 60, 70 or 80° C. for 30 min. After the solutions were cooled down to room temperature, the enzymatic (catalytic) activity of the complexed compounds of DNA with platinum complex and the horseradish peroxidase was measured similarly to Example 2. The results are shown in FIG. 7. The enzymatic (catalytic) activity of the horseradish peroxidase is decreased markedly by heat treatment at higher temperatures, whereas the activity of the complexed compounds of DNA with platinum complex is maintained after heat treatment at higher temperatures.

### Example 7

Purified, complexed compounds of DNA with platinum complex were prepared similarly to Example 1 except using single stranded salmon testes DNA (D-7656, molecular weight 468,000, purchased from Sigma-Aldrich Japan, Ltd.) and a cisplatin solution (500 ppm, Briplatin, Bristol-Myers Company, cis-diaminedichloro platinum, PtCl₂(NH₃)₂) as a platinum complex solution. Specifically, 10 μL of a buffer solution at pH 7 (a phosphate buffer), at pH 9 (a borate buffer) or at pH 11 (a disodium hydrogenphosphate-sodium hydroxide buffer solution), 20 μL of the cisplatin solution, and 10 μL of the DNA solution were put into a 1.5-mL Eppendorf tube, which was in turn shaded with aluminum foil, and were subsequently reacted at pH 7, 9, or 11 at room temperature (25° C.) for 120 hours. Ethanol precipitation was performed similarly to Example 1 so as to prepare the purified, complexed compounds of DNA with platinum complex. The enzymatic (catalytic) activity of 0.5 nmol/L of the complexed compound of DNA with platinum complex (cisplatin) was measured similarly to Example 2. The results are shown in FIG. 7. For comparison, the enzymatic (catalytic) activity was also measured for 0.5 nmol/L of the purified, complexed compound of DNA with platinum complex (potassium tetrachloroplatinate (II) (K₂[PtCl₄]) which was prepared in Example 1 using single stranded salmon testes DNA (D-7656, molecular weight 468,000, purchased from Sigma-Aldrich Japan, Ltd.) under a condition of pH 9 for a reaction time of 72 hours, and the enzymatic (catalytic) activity of 0.5 nmol/L of the purified, complexed compound of DNA with platinum complex (potassium tetrachloroplatinate (II) (K₂[PtCl₄]) which was prepared in Example 1 using [A-G] 10-mer, where adenine [A] and guanine [G] were linked (purchased from Sigma-Aldrich Japan, Ltd., a product purified through a cartridge, 1.0 μmol each), under a condition of pH 9 for a reaction time of 72 hours. The results are shown in Table 2. It is evident that the enzymatic (catalytic) activity was exhibited by the complexed compound of DNA with platinum complex that was prepared using not only potassium tetrachloroplatinate (II) (K₂[PtCl₄]), but also cisplatin.

TABLE 2
Catalytic activity of 0.5 nmol/L of DNA-platinum
complexes prepared under the various conditions.

| | | | | Reac- |
|---|---|---|---|---|
| | | Platinum | Reaction | tivity |
| Sample | Nucleic acid | complex | pH | (unit/L) |
| DNA-platinum | Single stranded | cisplatin | 7 | 0.043 |
| complex No. 6 | salmon testes DNA | | | |
| DNA-platinum | Single stranded | cisplatin | 9 | 0.190 |
| complex No. 7 | salmon testes DNA | | | |
| DNA-platinum | Single stranded | cisplatin | 11 | 0.136 |
| complex No. 8 | salmon testes DNA | | | |
| DNA-platinum | Single stranded | K₂[PtCl₄] | 9 | 1.768 |
| complex No. 9 | salmon testes DNA | | | |
| DNA-platinum | [A-G] 10-mer | K₂[PtCl₄] | 9 | 0.151 |
| complex No. 10 | | | | |
| RNA-platinum | Yeast RNA | K₂[PtCl₄] | 9 | 0.184 |
| complex No. 1 | | | | |
| RNA-platinum | Yeast transfer RNA | K₂[PtCl₄] | 9 | 0.339 |
| complex No. 1 | | | | |

### Example 8

RNA from yeast (300-500 base pairs, Ambion Co. Ltd.) and transfer RNA from yeast (molecular weight 25000-30000, Ambion Co. Ltd.) were dissolved into pure water to prepare 1000 ppm RNA solutions. Purified, complexed compounds of RNA with platinum complex were prepared, similarly to Example 1, under a condition of pH 9 for a reaction time of 72 hours except using RNA solutions instead of DNA solutions. The catalytic (enzymatic) activity of the complexed compounds of RNA with platinum complex and the horseradish peroxidase was measured similarly to Example except using 0.5 nmol/L of the complexed compounds of RNA with platinum complex. For comparison, the enzymatic (catalytic) activity was also measured for 0.5 nmol/L of the purified, complexed compound of DNA with platinum complex that was prepared in Example 1 using single stranded salmon testes DNA (D-7656, molecular weight 468,000, purchased from Sigma-Aldrich Japan, Ltd.) under a condition of pH 9 for a reaction time of 72 hours, and 0.5 nmol/L of the purified, complexed compound of DNA with platinum complex that was prepared in Example 1 using [A-G] 10-mer, where adenine [A] and guanine [G] were linked ((AG)₁₀ total 20 mers, purchased from Sigma-Aldrich Japan, Ltd., a product purified through a cartridge, 1.0 μmol each), under a condition of pH 9 for a reaction time of 72 hours. The results are shown in Table 2. It is evident that not only the complexed compounds of DNA with platinum complex, but also the complexed compounds of RNA with platinum complex, have the enzymatic (catalytic) activity.

### Example 9

In each 2-mL plastic tube, 20 μL of a synthetic oligodeoxyribonucleotide (29-mer, 5′-TGAAGGCTTGAGTAAATTATTCCATCAT AG-3′)(5 μg/μL of a TE buffer), 5 μL of pure water, 70 μL of a borate buffer (pH 9.2), and 5 μL of cis-diaminedichloro platinum (PtCl₂(NH₃)₂) (0.2M, in dimethylsulfoxide solution) were added to prepare a 100-μL solution, which was incubated at various temperatures for various time periods: at 95° C. for 10 min, for 30 min, or 1 hour; at 80° C. for 10 min, 30 min, or 1 hour; at 60° C. for 1 hour; at 45° C. for 1 hour; at 37° C. for 1 hour. After the reaction, the purified DNA-platinum complexes were precipitated similarly to Example 1.

The DNA-platinum complexes obtained were dissolved in a TE buffer (pH 8) before use in this Example. In the case where the DNA-platinum complexes could not be dissolved in the TE buffer, the supernatants were obtained after centrifugation at 15,000 rpm for 10 min. One μL of the resultant DNA-platinum complex solutions and 199 μL of a TMB substrate solution containing hydrogen peroxide were mixed and then incubated at 37° C. for 30 min. The reaction was stopped with the same volume of 1M phosphoric acid. The absorbance of the yellow color change due to the catalytic reaction was measured on a spectrophotometer (WPA, Inc.). Similarly, 1 μL of the DNA-platinum complex solutions obtained and 199 μL of an ABTS (2,2′-azino di(3-ethylbenzothiazoline-6-sulfonic acid) ammonium salt) substrate solution containing hydrogen peroxide (Nacalai Tesque, Inc., code 14351-80) were mixed and then incubated at 37° C. for 30 min. ABTS is known as a substrate for peroxidase. The reaction was stopped with the same volume of oxalic acid. The absorbance of the green color change due to the catalytic reaction was measured on a spectrophotometer (WPA, Inc.).

Similar experiments were also performed for a synthetic oligodeoxyribonucleotide with a contiguous CT sequence (5′-(CT)₂₉T-3′, 30-mer, custom order synthesis, Sigma-Aldrich, Japan, a product purified through a cartridge).

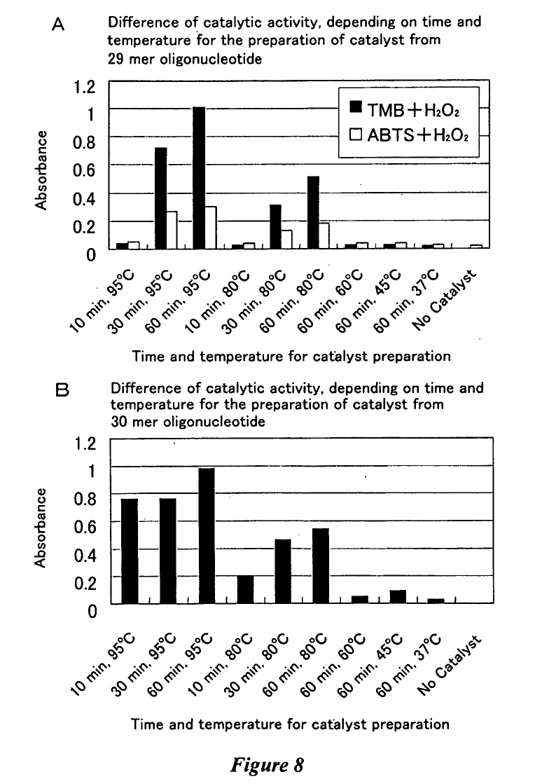

The results are shown in FIG. 8. It is found that both TMB and ABTS are substrates for the DNA-platinum complexes (FIG. 8A). The DNA-platinum complex prepared by the reaction at 95° C. for 1 hour exhibits high catalyst activity (FIG. 8B).

In the case of the above CT oligomer, all the DNA-platinum complexes that were prepared under the above conditions were soluble in the TE buffer. On the other hand, in the case of the above 29-mer, all the reaction products prepared at 95° C. were soluble in the TE buffer, although for those prepared at 80° C., a tiny amount of insoluble matter was found. For the products prepared at 45° C. and 60° C., insoluble matter was found, a large amount of precipitate was obtained after centrifugation, and little change in absorbance was observed after the supernatant was reacted with the substrate.

### Example 10

A synthetic oligodeoxyribonucleotide (5′-AGAGAGA-3′) (7-mer, custom order synthesis, Sigma-Aldrich Japan, LTD., a product purified through a cartridge) (2,179 μg) was dissolved in 200 μL of a TE buffer. To 25 μL aliquot of the solution, was added 70 μL of a borate buffer (pH 9.2), and 5 μL of cis-diaminedichloro platinum PtCl₂(NH₃)₂) (0.2M, in dimethylsulfoxide solution) or 5 μL of potassium tetrachloroplatinate (II) (K₂[PtCl₄]) (0.2 M aqueous solution). The mixed solutions were incubated in darkness at 80° C. for 2.5 hours. After the reaction, precipitation was performed with 3M sodium acetate and ethanol, similarly to Example 1. The precipitates were resuspended and washed in 70% ethanol, followed by centrifugation. The precipitates were dissolved in 200 μL of a TE buffer solution.

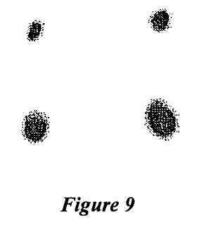

Two μL and 4 μL each of the resultant solutions were spotted on a nitrocellulose membrane (Millipore, Inc., HAWPO04700). The membrane was dried in air at room temperature for 5 min, and then at 80° C. for 30 min. This membrane was put in a Petri dish and subjected to staining with a staining solution containing naphthol derivative/benzidine derivative+hydrogen peroxide aqueous solution (a peroxidase staining kit, Nacalai Tesque, Inc., code 26652-70) at room temperature. Finally, after washing with water, the membrane was dried and photographed. Either spots due to the DNA-platinum complexes gave a red-purple color (FIG. 9).

### Example 11

Two DNA-platinum complexes were prepared. One was the DNA-platinum complex prepared using amino linker-modified DNA, the other is the DNA/platinum complex prepared using a cDNA of a protein as a material.

For the former, a synthetic oligonucleotide, the base sequence of which was a repeated sequence of adenine and guanine, and into which an amino linker (an aminohexyl NH₂(CH₂)₆— group) at the 5′-terminal (at the 5′-phosphate: 5′-amino linked-phosphate) had been introduced, 5′-amino linker (AG)₂₉ G-3′ (30-mer, custom order synthesis, Sigma-Aldrich Japan, Ltd., a product purified through a cartridge), was provided. 883 μg of the oligonucleotide was dissolved in 177 μL of a TE buffer solution. Twenty μL (100 μg) of the solution was treated at 95° C. for 7 min and then cooled rapidly on an ice bath. To this solution was added 140 μL of 0.05M of a borate buffer (pH 9.2), and 10 μL of cis-diaminedichloro platinum PtCl₂(NH₃)₂ (0.2M, in a dimethylsulfoxide solution). The mixture was incubated in darkness at 80° C. for 2.5 hours. After the reaction, precipitation was performed with 3M sodium acetate and ethanol, similarly to Example 1. The precipitates were resuspended and washed in 70% ethanol, followed by centrifugation. The precipitates were dissolved in 100 μL of a TE buffer solution. The soluble components transferring into the supernatant after centrifugation at 15,000 rpm for 10 min was used, as amino linker-modified DNA (AG30) platinum complex, for the following experiments.

For the latter, the DNA-platinum complex was prepared using a cDNA fragment of SCFb (stem cell factor precursor protein) as a material, as described below. A recombinant plasmid was obtained, which had been prepared by subcloning of a commercially available cDNA (I.M.A.G.E) of SCFb into pSP73 vector. Using this plasmid as a template, a SCFb cDNA fragment (corresponding to the amino-terminal fragment at positions 1 to 187) was obtained by PCR (polymerase chain reaction), as follows. A synthetic oligodeoxyribonucleotide containing Kpn site (33-mer, 5′-GGGGTACCATGAA GAAGACACAAACTTGGATTC-3′) was prepared according to the sequence information of SCFb cDNA. One hundred μM solution in a TE buffer was used as primer 1. A synthetic oligodeoxyribonucleotide containing BamHI site (33-mer, 5′-CGGGATCCAGCCACAATTACACTTCTTGAAAC-3′) was prepared according to the sequence information of SCFb cDNA. One hundred μM solution in a TE buffer was used as primer 2.

To each of 12 tubes for PCR, was added 22 μL of pure water, 25 μL of a PCR reaction solution (Promega, Inc., M7122), and 1 μL each of the template, primer 1 and primer 2. Amplification was performed on an amplifier, LittleGene (BioFlux), under the following conditions. After the treatment at 95° C. for 2 min, 40 cycles (at 95° C. for 1 min, at 55° C. for 1 min, and at 72° C. for 1 min) was run, followed by a reaction at 72° C. for 5 min, finally. The resultant reaction solution was subjected to phenol treatment and ethanol precipitation, which are routinely used for DNA treatment. Then, the precipitates were dissolved in a TE buffer. The cDNA fragment of SCFb (corresponding to the amino-terminal fragment at positions 1 to 187) was obtained.

Next, this 100-μL solution of the SCFb cDNA fragment (corresponding to the amino-terminal fragment at positions 1 to 187) (59 μg) was treated at 95° C. for 7 min, and cooled rapidly on an ice bath. To this, was added 90 μL of a borate buffer (pH 9.2) and 10 μL of cis-diaminedichloro platinum PtCl₂(NH₃)₂ (0.2M, in a dimethylsulfoxide solution) and then they were reacted at 80° C. for 2.5 hours. To the reaction solution, was added 20 μL of 3M sodium acetate solution and 500 μL ethanol and the mixture was kept at −20° C. for 1 day and then centrifuged at 15,000 rpm for 30 min. The resultant precipitates were washed with 70% ethanol, and then dissolved in a TE buffer, to obtain the DNA (SCFb cDNA)-platinum complex.

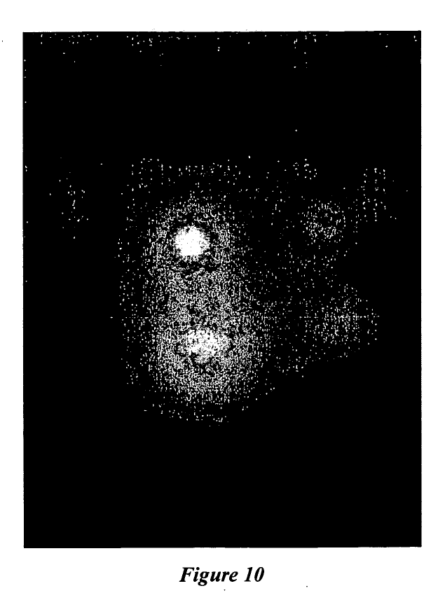

Two μL and 4 μL each of the two DNA-platinum complexes, that is, the amino linker-modified DNA (AG30) platinum complex and the DNA (SCFb cDNA)-platinum complex, were spotted on a nitrocellulose membrane (Millipore, Inc., HAWPO04700). The membrane was dried in air for 5 min, and then treated at 80° C. for 30 min. This membrane was dipped in 10 ml of a luminol reagent containing luminol and hydrogen peroxide (Santa Cruz Inc.; sc-2048). The filter was taken out, and then most of the reagent solution was wiped off. It was wrapped with a polyethylene wrap film, and brought into contact with a Polaroid 667 film for 1 min in darkness. White spots were obtained (FIG. 10).

### Example 12

It was investigated whether the use of a DNA labeled with a DNA-platinum complex allow to detect DNA with the specific sequence.

First, a DNA labeled with a DNA-platinum complex was prepared as follows. A recombinant plasmid, which had been prepared by subcloning of a commercially available cDNA (I.M.A.G.E) of SCFb into pSP73 vector, was obtained and used for the following experiments. By using this plasmid, into which the SCFb cDNA fragments was incorporated, as a template, double stranded cDNA fragment (corresponding to the amino-terminal fragment at positions 1 to 187), into one strand of which amino linker was introduced at the 5′ terminal, was prepared by PCR as follows. A synthetic oligodeoxyribonucleotide (5′—NH₂-GAAGGGATCTGCAGGAATCGTG-3′, a 22-mer modified with amino linker at 5′ terminal, custom order synthesis, Sigma-Aldrich Japan, Ltd., a product purified through a cartridge) was prepared according to the sequence information. One hundred μM solution in a TE buffer was used as primer 1. Synthetic oligodeoxyribonucleotide containing BamHI site (33-mer, 5′-CGGGATCCAGCCACAATTACACTTCTTGA AAC-3′) was prepared according to the sequence information of SCFb cDNA. One hundred μM solution in a TE buffer was used as primer 2.

To each of 12 tubes for PCR, was added 22 μL of pure water, 25 μL of a PCR reaction solution (Promega, Inc., M7122), and 1 μL each of the above template, primer 1 and primer 2. Amplification was performed on an amplifier, LittleGene (BioFlux), under the following conditions. After the treatment at 95° C. for 2 min, 40 cycles (at 95° C. for 1 min, at 55° C. for 1 min, and 72° C., for 1 min) was run, followed by a reaction at 72° C. for 5 min, finally. The resultant reaction solution was subjected to phenol treatment and ethanol precipitation, which are routinely used for DNA treatment. Then, the precipitates were dissolved in a TE buffer. The amino linker-modified SCFb cDNA fragment (corresponding to the amino-terminal fragment at positions 1 to 187) was obtained.

Next, to 48-μL solution of the amino linker-modified SCFb cDNA fragment (100 μg) was added 47 μL of a borate buffer (pH 9.2) and 5 μL of cis-diaminedichloro platinum PtCl₂(NH₃) 2 (0.2M, in a dimethylsulfoxide solution) and then they were reacted at 95° C. for 40 min. To the reaction solution was added 10 μL of 3M sodium acetate and 250 μL ethanol, and the mixture was kept at −20° C. for 30 min and then centrifuged at 15,000 rpm for 30 min. The resultant precipitates were washed with 70% ethanol, and then dissolved in 100 μL of a TE buffer, to obtain the DNA-platinum complex.

To 60 μL of this DNA-platinum complex was mixed 30 μL of the amino linker-modified SCFb cDNA fragment and then 15 μL of glutaraldehyde (2.5%). They were reacted at 37° C. for 20 min, and then treated at 95° C. for 5 min, and subsequently cooled rapidly on an ice bath, to obtain the SCFb cDNA fragment labeled with the DNA-platinum complex (DNA-platinum complex probe).

Next, 4 cDNAs were prepared: a cDNA fragment of human SCFb (official symbol: KITLG) (corresponding to the amino-terminal fragment at positions 1 to 187); a cDNA fragment of human NOV (nephroblastoma overexpressed gene) (official symbol: NOV) (corresponding to the amino-terminal fragment at positions 1 to 133); a cDNA fragment of human MFAP4 (microfibrillar-associated protein 4) (official symbol MFAP4) (corresponding to the amino-terminal fragment at positions 21 to 154); a cDNA fragment of human Delta1 (official symbol DLL1) (corresponding to the amino-terminal fragment at positions 1 to 168).

The cDNA fragments were obtained by PCR under the above PCR conditions using primers containing restriction enzyme sites, after subcloning from the commercially available cDNAs into pSP73 vectors. These 4 cDNA fragments were incubated at 95° C. for 5 min and then cooled rapidly on an ice bath to obtain their single stranded DNAs. The single stranded cDNAs were spotted at 300 ng/2 μL per spot on a cation-charged nylon membrane Hybond N+ (GE healthcare, Inc.; RPN82B), which had been cut into 2-cm square. After drying in air, the membrane was placed on a hot plate at 80° C. for 4 hours to immobilize the DNA thereon.

This membrane was pre-incubated with 1 mL of a commercially available DNA hybridization solution (Nacalai Tesque, Inc., code 04376-64) at 68° C. for 2 hours in a sealed plastic bag (Toyobo S-1001), which was floated in a water bath. Next, the hybridization solution in the bag was discarded and replaced with a fresh DNA hybridization solution. Then, in the plastic bag, 10 μL of the above DNA-platinum complex probe was added and mixed with the fresh DNA hybridization solution. DNA hybridization was conducted at 68° C. for 10 hours. Next, the membrane was washed with 0.1% SDS (sodium dodecyl sulfate) and 2×SSC (sodium chloride, sodium citrate solution, pH 7.0) at 68° C. for 15 min, 4 times. The washed membrane was stained with a staining solution containing naphthol derivative/benzidine derivative+hydrogen peroxide aqueous solution (peroxidase staining kit, Nacalai Tesque, Inc., code 26652-70) at room temperature. After washing with water, the membrane was dried and photographed.

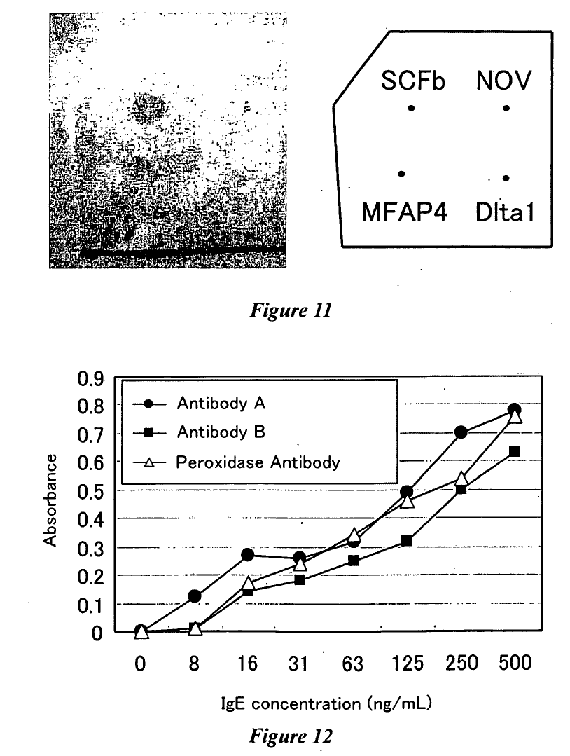

Due to catalytic reaction of DNA-platinum complex probe, the SCFb cDNAs were specifically detected as pale red-purple spots within 4 spots on the above nylon membrane (FIG. 11).

### Example 13

Sandwich ELISA (enzyme-linked immunosorbent assay) was conducted to confirm whether an antibody labeled with a DNA-platinum complex were able to detect its antigen.

First, a synthetic oligonucleotide, the base sequence of which was a repeated sequence of adenine and guanine, and into which an amino linker at the 5′-terminal had been introduced, 5′-amino linker (AG)₂₉ G-3′ (30-mer, custom order synthesis, Sigma-Aldrich Japan, Ltd., a product purified through a cartridge), was provided. 883 μg of the oligonucleotide was dissolved in 177 μL of a TE buffer solution. Twenty μL (100 μg) of the solution was treated at 95° C. for 7 min and then cooled rapidly on an ice bath. To this solution was added 140 μL of 0.05M of a borate buffer (pH 9.2), and 10 μL of cis-diaminedichloro platinum PtCl₂(NH₃)₂ (0.2M, in a dimethylsulfoxide solution). The mixture was incubated in darkness at 80° C. for 2.5 hours. After the reaction, precipitation was performed with 3M sodium acetate and ethanol, similarly to Example 1. The precipitates were resuspended in and washed with 70% ethanol, followed by centrifugation. The precipitates were dissolved in 100 μL of a TE buffer solution. The soluble components transferring into the supernatant after centrifugation at 15,000 rpm for 10 min was used, as amino group-introduced DNA-platinum complex, for the following experiments.

Next, antibodies labeled with the DNA-platinum complex were prepared by two kinds of methods (antibody A and Antibody B).

For Antibody A, 20 μL of the above amino group-introduced DNA-platinum complex, 80 μL of 0.05M phosphate buffer solution (pH 6.8) and 0.1 mL of goat anti-human immunoglobulin E (IgE) antibody (1 mg/mL) (Bethyl Laboratories, Inc., A80-108A) were mixed, and to the mixture was added 0.1 mL of 2.5% glutaraldehyde. They were reacted at 37° C. for 10 min. Immediately after being left at room temperature for 30 min, the product was used in the experiment.

For the preparation of Antibody B, 20 μL of dextran (molecular weight 500,000, Sigma-Aldrich, D8802, 20% aqueous solution), 12 μL of 0.1 M sodium periodate aqueous solution and 48 μL of distilled water were mixed and left to stand at room temperature for 5 hours. Then, to the mixture was added 1 mL of a distilled water, and the resultant mixed solution was centrifuged at 3,000 rpm at room temperature for 1 hour using a centrifugal filter (Amicon, Inc., ultra-15, permeation molecular weight 100,000). To the solution remaining unfiltered, was added 250 μL. After another centrifugation, the remaining solution that had not been filtrated by the filter was collected (180 μL). Thus, most of the unreacted sodium periodate was removed. To the remaining solution were added 30 μL of the above amino group-introduced DNA-platinum complex, 30 μL of 50 mM carbonate buffer solution pH 9.6, and 0.25 mL of the goat anti-human immunoglobulin E (IgE) antibody (1 mg/mL) (Bethyl Laboratories, Inc., A80-108A), and they were reacted at room temperature for 8 hours to obtain Antibody B.

Antibody B was labeled with dextran, to which a lot of the DNA-platinum complexes were bound via its amino group.

Detection of human immunoglobulin E (IgE) was done by a sandwich ELISA using Antibody A, Antibody B, or peroxidase-labeled anti-human antibody (Bethyl Laboratories, Inc., A80-108P) as a labeled antibody.

First, the antibody recognizing human immunoglobulin E (IgE) (Bethyl Laboratories, Inc., A80-108A) was dissolved in a carbonate buffer solution pH 9.6, and the solution was added to each well in a 96-well microplate, which was left at room temperature for 1 hour to immobilize the antibody on the bottom surface. To each of the wells was added a blocking reagent (1% albumin-containing Tris-buffered saline pH 8.0), and the plate was left at room temperature for 30 min. After removing the blocking reagent, each well was washed 5 times with a washing solution (0.05% Tween 20-containing Tris-buffered saline pH 8.0). Then, to each well was added human IgE Calibrator (Bethyl Laboratories, Inc., RC80-108), which had been diluted to various concentrations with 1% albumin-containing Tris-buffered saline, pH 8.0. After being left at room temperature for 1 hour, the unbound human IgE was removed. The wells were washed 5 times with a washing solution (0.05% Tween 20-containing Tris-buffered saline, pH 8.0).

Next, the above antibodies labeled with the DNA-platinum complex were added to each well, and allowed to react at room temperature for 1 hour. As control, peroxidase-labeled anti-human antibody was reacted in a similar way. After washing 5 times with the above washing solution, peroxidase substrates (TMB and 0.01% hydrogen peroxide solution) was added to each well and allowed to react at room temperature. The reaction was stopped by addition of 1M phosphoric acid. The absorbance of the yellow color change was measured on a spectrophotometer (WPA, Inc.). Detection by two labeled antibodies (Antibody A, and Antibody B) was comparable to, or higher in sensitivity than, that by the peroxidase-labeled antibody (FIG. 12).

This application relates to Japanese Patent Application No. 2006-122956, filed on Apr. 27, 2006.

The contents of all patents, patent applications and references cited in the present specification are incorporated herein in its entirety by reference, as if fully and specifically set forth herein, to the fullest extent permitted by applicable law.

1. An oxidation catalyst comprising a complex of a transition metal and a monomeric or polymeric nucleotide or an analogue thereof.

2. The catalyst according to claim 1, which exhibits a peroxidase-like oxidation catalytic activity.

3. The catalyst according to claim 1, which exhibits 1/100 (0.01) or more times the oxidation catalytic activity of horseradish peroxidase (activity: 1,000 units/mg) in an equimolar amount.

4. The catalyst according to claim 1, wherein the transition metal is selected from the platinum group consisting of ruthenium, rhodium, palladium, osmium, iridium and platinum.

5. The catalyst according to claim 1, wherein the transition metal is platinum or palladium.

6. The catalyst according to claim 5, wherein the transition metal is platinum.

7. The catalyst according to claim 6, wherein in the complex, the platinum is associated with the nucleotide or the analogue thereof, in the form of —PtCl2-, —PtCl3, —PtCl(H2O+)-, —Pt(H2O+)2—, —PtCl2(H₂O+), —PtCl(H2O+)2, —Pt(H2O+)3, —PtCl(NH3)2, —Pt(NH3)2- or —Pt(H2O+)(NH3)2.

8. The catalyst according to claim 1, wherein the polymeric nucleotide is a single-stranded nucleic acid.

9. The catalyst according to claim 1, wherein the polymeric nucleotide has at least 5 consecutive nucleotides.

10. The catalyst according to claim 1, wherein the polymeric nucleotide has at least 7 consecutive nucleotides.

11. The catalyst according to claim 1, wherein the nucleotide is a deoxyribonucleotide.

12. A peroxidase-like oxidation catalyst comprising a complex obtained by mixing in darkness a monomeric or polymeric nucleotide or an analogue thereof and a complex of a metal selected from the platinum group, in an aqueous reaction medium selected from phosphate buffer solutions, borate buffer solutions and disodium hydrogenphosphate-sodium hydroxide buffer solutions, under a neutral to alkaline condition.

13. The catalyst according to claim 12, wherein the complex of the metal selected from platinum group is a platinum complex.

14. The catalyst according to claim 13, wherein the platinum complex is potassium tetrachloroplatinate (II) or cis-dichlorodiamine platinum (II).

15. The catalyst according to claim 12, wherein the neutral to alkaline condition is at pH 7-11.

16. The catalyst according to claim 12, wherein the mixing is conducted at a temperature of 25-37° C. for 24-120 hours.

17. The catalyst according to claim 12, wherein the mixing is conducted at a temperature of 80-95° C. for 0.5-3 hours.

18. A reagent for detecting or quantifying a target substance for detection, wherein an substance capable of specifically binding the target substance for detection is associated with the catalyst according to claim 1 as a label, directly or via a linker.

19. The reagent according to claim 18, wherein the substance capable of specifically binding the target substance for detection is an antibody or an antibody fragment.

20. The reagent according to claim 18, wherein the substance capable of specifically binding the target substance for detection is a nucleotide probe.

21. A kit for detecting or quantifying a target substance for detection, comprising the reagent according to claim 18.

22. A method for detecting the presence of a target substance for detection in a sample, comprising contacting the sample with the reagent according to claim 18, and determining the presence/absence of the oxidation catalytic activity after removing any unreacted reagent.

23. A method for quantifying an amount of a target substance for detection in a sample, comprising contacting the sample with the reagent according to claim 18, measuring the oxidation catalytic activity after removing any unreacted reagent, and comparing the measured value with a predetermined standard curve.

24-25. (canceled)

26. A method for preparing an oxidation catalyst comprising a monomeric or polymeric nucleotide or an analogue thereof associated with a complex of a transition metal, the method comprising reacting in darkness the monomeric or polymeric nucleotide or the analogue thereof with the complex of the transition metal in an aqueous reaction medium under a neutral to alkaline condition, and collecting a reaction product.

27. The method according to claim 26, wherein the neutral to alkaline condition is at pH 7-11.

28. The method according to claim 26, wherein the collecting is by ethanol precipitation of DNA.

29. The catalyst of claim 2, wherein the transition metal is selected from the platinum group consisting of ruthenium, rhodium, palladium, osmium, iridium and platinum.

30. The catalyst of claim 2, wherein the transition metal is platinum.
